(19) United States
(12) Patent Application Publication
(10) Pub. No.: US 2024/0142449 A1
(43) Pub. Date: May 2, 2024

(54) METHOD FOR DETERMINING THE PRESENCE OF A TARGET MICROORGANISM IN A BIOLOGICAL SAMPLE

(71) Applicant: INOVA BIOMEDICAL TECHNOLOGY S.R.L., CATANIA (IT)

(72) Inventors: Sabrina Conoci, CATANIA (IT); Francesco Traina, CATANIA (IT); Salvatore Guglielmino, CATANIA (IT)

(21) Appl. No.: 18/548,721

(22) PCT Filed: Mar. 8, 2022
(86) PCT No.: PCT/IB2022/052050

§ 371 Date: Sep. 1, 2023

(30) Foreign Application Priority Data
Mar. 8, 2021 (IT) 102021000005363

(51) Int. Cl. G01N 33/569; G01N 33/543; G01N 33/58

(52) U.S. Cl. CPC G01N 33/56938; G01N 33/54388; G01N 33/582; G01N 2333/31

(57) ABSTRACT

The present invention relates to a method for determining the presence of a target microorganism in a biological sample comprising the steps of:
providing a strip made of porous material, said strip having at least one fixation zone on which at least one phage exposing a peptide selective for said microorganism is fixed, and a deposition zone, separated from said fixation zone and intended to receive a portion of said biological sample, said phage being bound to a marker in deactivated form;
contacting said biological sample with said strip on said deposition zone and eluting said microorganism through said strip so that said microorganism reaches said fixation zone to form a phage-target microorganism complex and release said marker in activated form;
detecting said marker in activated form.

### CROSS-REFERENCE TO RELATED APPLICATIONS

This patent application claims priority of Italian Patent Application No. 102021000005363 filed on Mar. 8, 2021, the entire disclosure of which is incorporated herein by reference.

### TECHNICAL FIELD

The present invention relates to methods for determining the presence of a target microorganism in a biological sample.

### STATE OF THE ART

Because of increased life expectancy, the number of prosthetic replacements continues to grow at a significant rate, with several million prosthetic joints implanted each year worldwide. Most of the time, this type of surgical procedure has positive results with improved joint function and pain relief. However, periprosthetic infection (PJI) is one of the complications that can occur, with an incidence ranging from 2.0% to 2.4% for primary prosthetic interventions, but increasing up to 20% for prosthetic revision procedures.

This high incidence leads to a true economic burden on world health services, since the costs for treating a PJI are very high.

This event creates significant clinical and economic problems due to the high associated costs. In fact, following the onset of a PJI, patients are treated with a complex clinical process that can last more than a year for the most serious cases and which provides for:
Explanation of the prosthesis and biopsy sampling (surgical eradication);
Diagnosis of the infectious agent on the biopsy sample (bacterial culture—about 2 weeks);
Temporary prosthesis implant (antibiotic-loaded spacer);
Therapeutic treatment (1-3 months);
Final prosthesis implantation after revision with negative biohumoral parameters and after a biopsy with negative antibiogram.

Furthermore, if the parameters do not normalize, the surgical procedure for spacer cleaning and replacement is repeated until the infection is eradicated.

In addition to representing a burdensome clinical path for the patient, such process also induces consequent negative repercussions for its emotional consequences, as well as on healthcare costs, including burdensome costs of hospitalization, of diagnostics, of antibiotic therapy, of treatment and additional prosthetic material, all deriving from the septic complication of prostheses.

Such scenario is a consequence of the fact that currently there are no specific diagnostic tools on the market that allow early, rapid, and low-cost diagnoses.

As regards the diagnostic part, it provides for complex and not always decisive procedures that include biopsy sampling, clinical blood tests, and advanced magnetic resonance imaging. Cultures, biopsies, serological markers of inflammation, and imaging techniques all have advantages and disadvantages. For example, dosage of C-reactive protein (PCR), erythrocyte sedimentation rate (ESR), and leukocyte count are not sensitive or sufficiently specific for detecting or excluding a periprosthetic infection (PJI). Joint aspiration (arthrocentesis) entails a risk of infection and its sensitivity is very variable.

In general, for a periprosthetic infection diagnosis, a combination of clinical, laboratory, microbiological and imaging analyses is required, performed on the basis of the personal experience of the clinician and of the available techniques and of the financial resources of each individual hospital unit.

US2009286225 describes a method for detecting bacteria in a sample by using bacteriophages.

Bacteriophages are organisms that have evolved in nature in order to exploit bacteria for replicating. In particular, the phage attaches itself to the bacterium and injects its own DNA into it, inducing it to replicate the phage hundreds of times. At the end of replication, some bacteriophages also cause the bacterium to lyse in order to infect new bacteria. The estimated time for phage attachment, its incubation, replication, and amplification may even require several hours.

Therefore, the method of US2009286225, which requires the amplification of the bacteriophage by incubation, requires a long time before being able to perform the detection of the possible presence of bacteria. Therefore, such process is not suitable for a use thereof in the operating room during the surgical procedure.

The need is thus felt in the art for new, rapid, cost-effective diagnostic methods for infections caused by microorganisms and devoid of the disadvantages of the known art.

### Subject of the Invention

Such object of the present invention is achieved by means of methods for determining the presence of a target microorganism according to claims 1, 2 and 3.

In particular, according to a first aspect of the invention, a method is provided for determining the presence of a target microorganism in a biological sample comprising the steps of:
contacting said biological sample with a phage exposing a peptide selective for said microorganism, said phage being bound to a marker to form a phage-marker complex;
letting said phage react with said biological sample so as to allow, if said microorganism is present, the binding of said phage to said target microorganism to obtain a sample comprising a microorganism-marked phage complex;
filtering said sample comprising a microorganism-marked phage complex on a filter capable of retaining said microorganism-marked phage complex;
detecting said microorganism-marked phage complex.

Advantageously, the use of markers allows performing the step of detecting the presence of the microorganism without requiring the replication and amplification of the bacteriophage.

In fact, after filtration, the retained microorganism-marked phage complex emits a detectable signal directly on the filter.

A second aspect of the invention further provides a method for determining the presence of a target microorganism in a biological sample comprising the steps of:
contacting a marker with said biological sample to obtain, if said microorganism is present, a target microorganism-marker complex;
providing a strip made of porous material, said strip having at least one fixation zone on which at least one phage exposing a peptide selective for said microorganism is fixed, and a deposition zone, separated from said fixation zone and intended to receive a portion of said target microorganism-marker complex;
contacting said target microorganism-marker complex with said strip on said deposition zone and eluting said complex through said strip so that said complex reaches said fixation zone to form a phage-target microorganism-marker complex;
detecting said phage-target microorganism-marker complex.

In one embodiment, when the marker is used to mark the phage, it is selected from the group consisting of fluorescent markers, such as rhodamine, fluorescein isothiocyanate, 4′,6-diamidin-2-phenylindole, Cyto9, Cyto5, colorimetric markers, electrochemical markers such as ferrocene, and magnetic markers, such as ferric oxide (Fe₂O₃) or chromium dioxide (CrO₂) nanoparticles. Preferably, the marker is a cellular dye such as 4′,6-diamidin-2-phenylindole (DAPI), Cyto9, Cyto5. When the marker is used to mark the microorganism, it is selected from the group consisting of fluorescent molecular systems such as DAPI (4′,6-diamidin-2-phenylindole), Cyto9, Cyto5, rhodamine, fluorescein isothiocyanate, optionally conjugated to magnetic nanoparticles such as ferric oxide (Fe₂O₃) or chromium dioxide (CrO₂) nanoparticles, electrochemical molecular systems such as ferrocene.

According to a third aspect of the invention, a method is finally provided for determining the presence of a target microorganism in a biological sample comprising the steps of:
providing a strip made of porous material, said strip having at least one fixation zone on which at least one phage exposing a peptide selective for said microorganism is fixed, and a deposition zone, separated from said fixation zone and intended to receive a portion of said biological sample, said phage being bound to a marker in deactivated form;
contacting said biological sample with said strip on said deposition zone and eluting, if present, said microorganism through said strip so that said microorganism reaches said fixation zone to form a phage-target microorganism complex and release said marker in activated form;
detecting said marker in activated form.

In this case the marker is selected from the group consisting of carbon dots, semiconductor nanoparticles such as SeC or fluorophore molecular systems such as phenylbutazone.

According to the present invention, the term “phage” includes a non-lytic engineered bacteriophage and refers to a virus that can attack a viable bacterium or other microscopic organisms and uses them to replicate.

Since the methods of the invention do not require the replication and amplification of the phage to be able to pass to the detection step, said methods are particularly rapid and suitable to be used for identifying the presence of bacteria in a sample already in the operating room during the surgical procedure for prosthesis application. Furthermore, thanks to the use of phages exposing sequences selective for a specific target microorganism, they are particularly precise methods for identifying the specific infection.

The methods of the invention are particularly useful for identifying, in a biological sample, target microorganisms selected from the group consisting of Pseudomonas aeruginosa, Staphilococcus aureus, Escherichia coli and Staphilococcus epidermidis.

Furthermore, the peptide selective for the microorganism is preferably selected from peptides having a peptide sequence selected from the group consisting of SEQ ID No.1, SEQ ID No.2, SEQ ID No.3 and SEQ ID No.4.

In particular, it has been found that phages exposing the peptide sequence identified as SEQ ID No. 1 are selective for Pseudomonas aeruginosa, phages exposing the peptide sequence identified as SEQ ID No. 2 are selective for Staphilococcus aureus, phages exposing the peptide sequence identified as SEQ ID No. 3 are selective for Escherichia coli, and phages exposing the peptide sequence identified as SEQ ID No. 4 are selective for Staphilococcus epidermidis.

### BRIEF DESCRIPTION OF THE DRAWINGS

The invention will now be described with reference to the figures, wherein:

FIG. 1 shows a first method according to the invention;

FIG. 2 shows a second method according to the invention;

FIG. 3 shows a third method according to the invention;

FIG. 4 shows the emission spectrum obtained with the detection of P. aeruginosa according to Example 3.

### DETAILED DESCRIPTION OF PREFERRED EMBODIMENT OF THE INVENTION

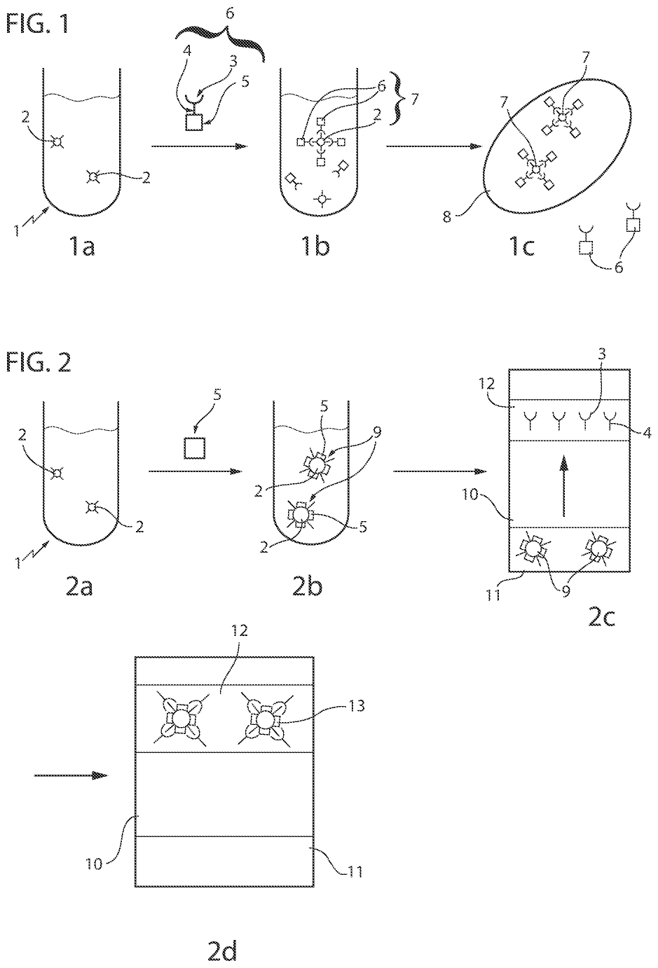

With reference to FIG. 1, reference numeral 1 indicates a biological sample comprising microorganisms 2, for example Staphilococcus aureus. Such sample, in the form of a solution, is contacted with the complex 6 containing a phage 4 exposing a peptide 3 selective for S. aureus, for example a phage exposing a peptide having the peptide sequence of SEQ ID No.2, and bound to a marker 5. In one embodiment, the marker 5 consists of a fluorochrome such as rhodamine, fluorescein isothiocyanate, 4′,6-diamidine-2-phenylindole, or an electrochemical marker such as ferrocene or a magnetic marker such as ferric oxide (Fe₂O₃) or chromium dioxide (CrO₂) nanoparticles. A microorganism-marked phage complex 7 is thus formed (FIG. 1b), which has such dimensions as to be able to be retained by a filter 8 having pore sizes of 0.22 to 0.45 microns. Once filtered, the microorganism-marked phage complex 7 present on the filter 8 (FIG. 1c) is detected with known optical detection methods, for example fluorescence microscopy or optical systems that read fluorescence, made of a light emitter (LED) and of a detector such as a photodiode, with electrochemical detection methods such as 3-electrode devices, with magnetic detection methods such as magnetic reading devices.

In an alternative embodiment of the invention, depicted in FIG. 2, the biological sample 1, in the form of a solution, comprising microorganisms 2 (FIG. 2a), for example S. aureus, is contacted with a marker 5 to create a target microorganism-marker complex 9 (FIG. 2b). In this method, the detection of the microorganism 2 possibly present in the biological sample 1, is carried out on a strip 10 of porous material, for example made with porous paper, microstructured polymers or sintered polymers. These devices are commonly known in the art as lateral flow devices and are able to spontaneously transport fluids.

The strip 10 is provided with a deposition zone 11, defining a deposition area, intended to receive a portion of the target microorganism-marker complex 9, preferably placed at one end of the strip 10, and a fixation zone 12, separated from the deposition zone 11 and defining a fixation area, on which phages 4 selective for S. aureus are immobilized, for example a phage 4 exposing a peptide 3 having the peptide sequence of SEQ ID No.2.

Immobilization of the phage in the fixation zone 12 takes place through known methods, for example by deposition and drying at room temperature.

The strip 10 is then contacted with the solution containing the target microorganism-marker complex 9. The solution then flows along the strip 10 in the direction of the arrow (FIG. 2c) until it reaches the fixation zone 12, where the target microorganism-marker complex 9 reacts with the phage 4 selective for such complex, for example a phage exposing a peptide 3 having the peptide sequence of SEQ ID No.2, to form a phage-target microorganism-marker complex 13.

Subsequently, the detection of the phage-target microorganism-marker complex 13 is carried out.

In one embodiment, the strip 10 may comprise multiple fixation zones 12 on each of which phages selective for a different microorganism are fixed. In this way it is possible to detect the presence of multiple, different microorganisms in a same biological sample.

In one embodiment, the marker 5 consists of fluorescent molecular systems, such as DAPI (4′,6-diamidin-2-phenylindole), Cyto9, Cyto5, rhodamine, fluorescein isothiocyanate, optionally conjugated with magnetic nanoparticles such as ferric oxide (Fe₂O₃) or chromium dioxide (CrO₂) nanoparticles, electrochemical molecular systems such as ferrocene, which mark the microorganism. The phage-target microorganism-marker complex 13 is detected with known optical detection methods, for example optical or fluorescence microscopy or optical systems that read fluorescence made of a light emitter (LED) and of a detector such as a photodiode, with electrochemical detection methods such as 3-electrode devices, with magnetic detection methods such as magnetic reading devices.

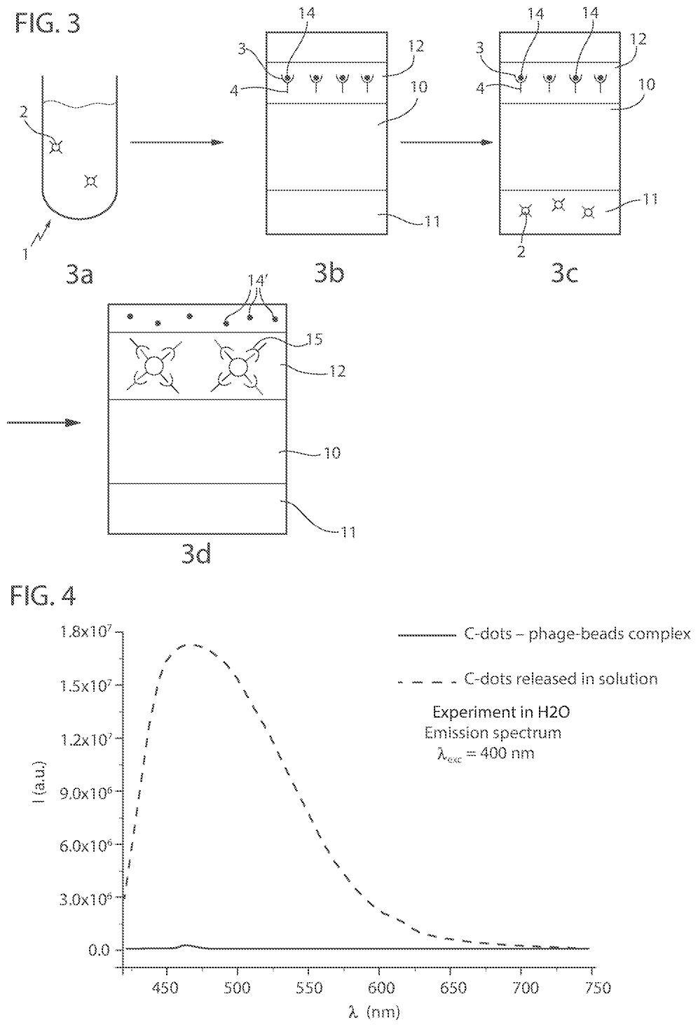

In an alternative embodiment of the invention, shown in FIG. 3, instead of resorting to the marking of the microorganism 2 possibly present in the biological sample 1, a marker 14 is used which, once bound to the phage 4 immobilized on the strip 10, is deactivated. Examples of such markers 14 are, for example, carbon dots. Alternative markers to carbon dots are semiconductor nanoparticles such as SeC or fluorophore molecular systems whose fluorescence is quenched by energy or electron transfer processes by means of the contact with the specific peptide expressed by the phage, such as phenylbutazone (PB). In particular, “deactivated marker” or “marker in deactivated form” means a marker which, following its binding to the phage, loses its ability to emit a signal detectable by standard instrumentation. On the contrary, the term “activated marker” or “marker in activated form” means a marker capable of emitting a signal detectable by standard instrumentation.

Once the target microorganism 2, possibly present in the biological sample 1, reaches the fixation zone, it binds the phage 4 causing the formation of the phage-target microorganism complex 15 and the breaking of the bond between the phage 4 and the marker 14. Such breaking causes the activation of the marker 14′ which will be able to be detected with standard instrumentation.

Alternatively, the phage bound to the deactivated marker can be immobilized on magnetic beads. The target microorganism, possibly present in the biological sample, contacted with the phage-deactivated marker-bead complex, binds to the phage causing the detachment of the marker and its activation.

Further characteristics of the present invention will result from the following description of some merely illustrative and non-limiting examples.

### Example 1

Step 1: Release of the Microorganisms Present from a Sample of Tissue or Synovial Fluid

Conditions:
Add at least 1 ml of sterile physiological solution (cover at least 90% of the sample).
Vortex the container with the sample for 30″.
Sonicate at 40 KHx 0.22+/−0.4 VVcm² for 5 minutes.
Vortex for 30″.

Step 2: Bacterium Marking

A volume of sonicated liquid is collected and 4′,6-diamidine-2-phenylindole (DAPI) dye for the bacterial cells is added.

Conditions:
Add 10 μl of DAPI stock solution (Sigma-Aldrich, Germany) (0.1 μg/ml in PBS) to 1 ml of the treated sample. The samples are incubated in the dark at 30° C. for 10-20 minutes under gentle stirring.

Step 3: Preparation of the Lateral Flow Device

Description of the Phage Probe

For the example shown, two libraries of M13 phage peptides (pVIII-9aa and pVIII-12aa) were used through the affinity selection described below. These libraries consist of M13 filamentous phages exposing random peptides of 9 or 12mer, respectively, fused to the main coat protein (pVIII). The libraries of nonapeptides and dodecapeptides were constructed in the vector pC89 (Felici et al., 1991), by cloning a random DNA insert between the third and the fifth codon which encode the mature pVIII (Luzzago and Felici, 1998).

Phage Peptide Selection

The screening of the library was performed using four rounds of affinity selection. The selection against P. aeruginosa whole cells was performed by incubating 10¹² phage particles with P. aeruginosa cells (OD660 0.5) in phosphate-buffered saline (PBS, 137 mM NaCl, 2.7 mM KCl, 10 mM phosphate buffer, pH 7.4; 1 ml) for 60 min at room temperature under gentle stirring. Bacteria and phages were precipitated by centrifugation for 5 min at 16,000×g, and the unbound phages were separated by a series of 10 washing and centrifugation steps (16,000×g, 5 min) with 1 ml TBS/Tween Buffer (Tris—50 mM HCl (pH 7.5), 150 mM NaCl, 0.05% (v/v) Tween 20). The phages bound to P. aerugonosa cells were pelleted with the cells and finally eluted with 200 nl of 0.2M glycine-HCl (pH 2.2) under gentle stirring at room temperature for 20 min, followed by neutralization with 150 nL of 1M Tris-HCl (pH 9.1). The phages eluted from the last round of affinity selection against P. aeruginosa were used to infect E. coli TG1 cells. Bacterial colonies, each containing phages from a single clone of the library, are randomly selected and propagated for subsequent affinity and specificity analyses. The phage DNA was extracted from the individual colonies of infected bacteria and used for PCR and sequencing. The sequencing primers are M13-40 reverse (5-GTTTTCCCAGTCACGAC-3, SEQ ID No. 5) and E24 forward (5-GCTACCCTCGTTCCGATGCTGTC-3, SEQ ID No. 6). The DNA sequences were translated into amino acids using the “translate” program on the proteomics server of the Swiss Institute of Bioinformatics Expert Protein Analysis System (ExPASy, http://www.expasy.ch/).

The phage clone, referred to as P9b, which showed the best binding capacity and specificity, exposed a peptide with the following peptide sequence: QRKLAAKLT, SEQ ID No. 1
Substrate of the lateral flow device: nitrocellulose—bio-Rad nitrocellulose membrane (0.2 μm pore)

Immobilization Method

On the test strip, the fixation zone is approximately 1 cm wide, and the amount of capture reagent (phage P9b) bound is 0.05-μg. The phage was bound by drying at room temperature.

Step 4: Detection

The solution containing the DAPI-marked bacterium is contacted with the lateral flow device in the deposition zone. The solution flows until it reaches the fixation zone where the phage probe specific for the microorganism was immobilized during the device preparation step: if present, said microorganism is captured by the specific probe and a presence transduction signal is detected in the zone specific for this microorganism.

Depending on the type of marking carried out on the microorganism, the signal is an optical signal of emission or absorption.

### Example 2

A) Starting Sample: Synovial Tissue or Fluid

Step 1: Release of the Microorganisms Present from a Tissue Sample

The tissue collected by the surgeon is sonicated in order to free any microorganisms present.

Conditions:
Add at least 1 ml of sterile physiological solution (cover at least 90% of the sample).
Vortex the container with the sample for 30″.
Sonicate at 40 KHx 0.22+/−0.4 VVcm² for 5 minutes.
Vortex for 30″.

Step 2: Bacterium Marking

A volume of sonicated liquid is collected and a solution containing the specific marked phage for recognizing the target microorganism is added.

Phage Probe Description

The phage probe is the same as in the previous example.

The phage clone, referred to as P9b, which exposes a peptide having the peptide sequence QRKLAAKLT (SEQ ID No.1), capable of specifically binding P. aeruginosa, was marked with fluorescein isothiocyanate according to the method described below.

Phage Marking with FITC

5×10¹⁰ PFU of phage P9b were resuspended in 200 ml of Na₂CO₃/NaHCO₃ buffer (pH 9.2) with 5 ml of fluorescein isothiocyanate (FITC, 5 mg/ml). The phages were incubated for 2 h in the dark on a rotating wheel at room temperature to allow the reaction with fluorochrome. The sample was incubated at 4° C. for 12 hours with 200 ml of PEG/NaCl, and then centrifuged twice at 15,300×g at 4° C. for 1 h. The supernatant was discharged, and the pellet was resuspended in 100 μl of Tris buffer solution [TBS (7.88 g/l Tris-HCl, 8.77 g/l NaCl)]. After marking, the phages were progressively dialyzed against 2 l of a TBS mixture (1:1) for 24 h. The marked phages were stored in the dark at 4° C. until use.

Step 3: Detection by Filtration

The solution containing the marked phage-microorganism complex, left to react for 30′ in the dark, is passed through a filter (black polycarbonate 0.45 n) which retains only the largest aggregates (marked phage-microorganism complex) and allows the smaller ones to go through (marked phage). As a result, the complexes thus obtained can be visualized on the filter.

### Example 3

Step 1: Phage Immobilization on Magnetic Beads

I. Functionalization of Tosyl-Activated Dynabeads M-280 with Phage Clone-pVIII Li2 (Specifically Binding P. aeruginosa ATCC 27853)

50 μl of tosyl-activated Dynabeads M-280 (Invitrogen cat. 142.03) are placed in a round bottom Eppendorf and washed twice as follows: with 500 μl of Borate Buffer (0.1 M Borate Buffer pH 9.5), for 5′ under gentle stirring on a wheel and 10′ on the magnet before discharging the supernatant. The beads are separated on the magnetic device for 10′, the buffer is discharged, and they are resuspended in 50 μl of Borate Buffer. 3×10⁶ phage clones Li2, i.e. 30 μl from the 1×10¹² TU/ml stock in buffer borate, were added, then 60 μl Borate Buffer and 60 μl of ammonium sulphate buffer (3M pH 7.4) were added. The tube was incubated for 24 h at 37° C. under gentle stirring on an inclined wheel.

The beads were separated on the magnetic device for 10′, the supernatant was discharged, and they were washed twice with 500 μl of PBS Buffer pH 7.4+1% BSA for 5′ on a wheel. Then, 500 μl of Blocking Buffer (PBS pH 7.4+4% BSA) were added and left on a wheel for 2 h at RT. The beads were separated on the magnetic device for 10′, the supernatant was discharged, and they were washed twice with 500 μl of PBS Buffer pH 7.4+1% BSA for 5′ on a wheel. Finally, the beads were resuspended in 200 μl of PBS and left at 4° C.

II. ELISA to Verify Dynabeads Functionalization with Li2

To verify that there were phages on the beads after this treatment, an ELISA test was carried out. 20 μl of beads are washed twice with 500 μl of Washing Buffer (PBS pH 7.4+0.5% Tween 20) for 5′ on a wheel. The beads were resuspended in 100 μl of anti-M13 HPR antibody (1:2500 in PBS+0.1% BSA+0.5% Tween 20) incubated on a wheel at 37° C. for 1 h. Then, 5 washings in 500 μl of 0.5% PBS Tween were carried out. In the last step, the beads were resuspended in 250 μl of TMB and incubated for 20′ on a wheel at RT, in the dark. Upon complete development, the reaction was stopped with 31 μl of 6N H₂SO₄. The beads were placed on the magnet for 10′, then 200 μl of the total solution and of a 1:10 dilution were read at 450 nm. The results obtained are shown in Table 1.

TABLE 1

Reading value at 450 nm

| | 200 μL of solution | 1:10 dilution |
|---|---|---|
| Dynabeads-Li2 | 2.415 | 1.407 |

Step 2: Complexation with Fluorescent Nanosystems (e.g. CDots-CD)

III. Dynabeads-Li2 Functionalization with CarbonDots (1.08 mg/ml)

CarbonDots extracted according to the method reported in Sawalha, S.; Silvestri, A.; Criado, A.; Bettini, S.; Prato, M.; Valli, L. Tailoring the sensing abilities of carbon nanodots obtained from olive solid wastes. Carbon, 2020, 167, 696-708, were used in a functionalization process with the Dynabeads-Li2.

Conditions of the tests performed in H₂O: 20 μl of Dynabeads-Li2 (approximately 2×10⁷ beads-Li2) prepared as described above, are washed twice on a wheel for 5′ with 500 μl of PBS. Then, they are placed on the magnet for 10′ and, having discharged the supernatant, are resuspended in 200 μl of H₂O. 800 μl of CarbonDots (1.08 mg/ml) are then added.

The samples are placed for 2 hours on an inclined wheel at 37° C. The Dynabeads-Li2-CDots complexes thus formed were separated on the magnet for 10′. Then, the post-functionalization supernatants are recovered, while the Dynabeads-Li2-CDots complexes are resuspended in 1 ml of H₂O, respectively.

Post-functionalization supernatants and Dynabeads-Li2-CDots complexes were analyzed by UV-vis and by fluorescence emission analysis.

Step 3: Detection of the Target Microorganism—P. aeruginosa Capture by Dynabeads-Li2-Cdots

In the presence of the target, the fluorescent molecule which is quenched by the interaction with the phage complex, is released into solution and the fluorescence reappears.

Experimental Conditions:

An o.n. culture of P. aeruginosa ATCC 27853 from isolation on Cetrimide agar plate in 5 ml of LB was prepared. The bacteria were collected by centrifugation at 8000×g for 10′ and resuspended in an isovolume of PBS.

The bacteria thus prepared were used to obtain a bacterial stock in PBS having a OD₆₀₀ of 0.29 (equal to 10⁸ cells/ml). The Dynabeads-Li2-Cdots complexes (previously analyzed by UV-Vi and fluorescence, then diluted in an overall volume of about 3 ml) were recovered with the aid of the magnet, then resuspended in 1 ml of 10⁶ cells/ml in PBS. The samples were placed on an inclined wheel at 37° C. for 30′.

The samples are first analyzed as such by UV-Vis and Fluorescence.

Then, the Dynabeads-Li2-CDots-P. aeruginosa complexes were separated on the magnetic device for 10′, the collected supernatant was centrifuged to remove any residual bacteria, then the post-capture supernatant was recovered. Both samples, in the two portions, Dynabeads-Li2-CDots-P. aeruginosa complexes and post-capture supernatant, are then analyzed by UV-Vis and fluorescence. The results are shown in FIG. 4.

1. A method for determining the presence of a target microorganism (2) in a biological sample (1) comprising the steps of:
providing a strip (10) made of porous material, said strip (10) having at least one fixation zone (12) on which at least one phage (4) exposing a peptide (3) selective for said microorganism (2) is fixed, and a deposition zone (11), separated from said fixation zone (12) and intended to receive a portion of said biological sample (1), said phage (4) being bound to a marker (14) in deactivated form;
contacting said biological sample (1) with said strip (10) on said deposition zone (11) and eluting, if present, said microorganism (2) through said strip (10) so that said microorganism (2) reaches said fixation zone (12) to form a phage-target microorganism complex (15) and release said marker (14′) in activated form;
detecting said marker (14′) in activated form.

2. Method for determining the presence of a target microorganism (2) in a biological sample (1) comprising the steps of:
contacting a marker (5) with said biological sample (1) to obtain, if said microorganism (2) is present, a target microorganism-marker complex (9);
providing a strip (10) made of porous material, said strip (10) having at least one fixation zone (12) on which at least one phage (4) exposing a peptide (3) selective for said microorganism (2) is fixed, and a deposition zone (11), separated from said fixation zone (12) and intended to receive a portion of said target microorganism-marker complex (9);
contacting said target microorganism-marker complex (9) with said strip (10) on said deposition zone (11) and eluting said complex (9) through said strip (10) so that said complex (9) reaches said fixation zone (12) to form a phage-target microorganism-marker complex (13);
detecting said phage-target microorganism-marker complex (13).

3. Method for determining the presence of a target microorganism (2) in a biological sample (1) comprising the steps of:
contacting said biological sample (1) with a phage (4) exposing a peptide (3) selective for said microorganism (2), said phage (4) being bound to a marker (5) to form a phage-marker complex (6);
letting said phage (4) react with said biological sample (1) so as to allow, if said microorganism (2) is present, the binding of said phage (4) to said target microorganism (2) to obtain a sample comprising a microorganism-marked phage complex (7);
filtering said sample comprising a microorganism-marked phage complex (7) on a filter (8) capable of retaining said microorganism-marked phage complex (7);
detecting said microorganism-marked phage complex (7).

4. Method according to claim 1, wherein said marker (13) is comprised of carbon dots, semiconductor nanoparticles such as SeC, or fluorophore molecular systems such as phenylbutazone.

5. Method according to claim 2, characterized in that said marker (5) is selected from the group consisting of fluorescent markers, colorimetric markers, electrochemical markers, and magnetic markers.

6. Method according to claim 2, characterized in that said marker (5) is selected from the group consisting of rhodamine, fluorescein isothiocyanate, 4′,6-diamidin-2-phenylindole, Cyto9, Cyto5, ferrocene, ferric oxide nanoparticles and chromium dioxide nanoparticles.

7. Method according to claim 3, characterized in that said marker (5) is selected from the group consisting of rhodamine, fluorescein isothiocyanate, 4′,6-diamidin-2-phenylindole, Cyto9, Cyto5 optionally bound to magnetic nanoparticles such as ferric oxide (Fe₂O₃) or chromium dioxide (CrO₂) nanoparticles, electrochemical molecular systems such as ferrocene.

8. Method according to claim 1 characterized in that said strip (10) has several fixation zones (12) on each of which a phage (4) exposing a peptide (3) selective for a microorganism (2) is fixed.

9. Method according to claim 1 characterized in that said target microorganism (2) is selected from the group consisting of Pseudomonas aeruginosa, Staphilococcus aureus, Escherichia coli and Staphilococcus epidermidis.

10. Method according to claim 1 characterized in that said peptide (3) is a peptide having a peptide sequence selected from the group consisting of SEQ ID No.1, SEQ ID No.2, SEQ ID No.3 e SEQ ID No.4.

11. Method according to claim 3, characterized in that said marker (5) is selected from the group consisting of fluorescent markers, colorimetric markers, electrochemical markers, and magnetic markers.

12. Method according to claim 2 characterized in that said strip (10) has several fixation zones (12) on each of which a phage (4) exposing a peptide (3) selective for a microorganism (2) is fixed.

13. Method according to claim 2 characterized in that said target microorganism (2) is selected from the group consisting of Pseudomonas aeruginosa, Staphilococcus aureus, Escherichia coli and Staphilococcus epidermidis.

14. Method according to claim 3 characterized in that said target microorganism (2) is selected from the group consisting of Pseudomonas aeruginosa, Staphilococcus aureus, Escherichia coli and Staphilococcus epidermidis.

15. Method according to claim 2 characterized in that said peptide (3) is a peptide having a peptide sequence selected from the group consisting of SEQ ID No.1, SEQ ID No.2, SEQ ID No.3 e SEQ ID No.4.

16. Method according to claim 3 characterized in that said peptide (3) is a peptide having a peptide sequence selected from the group consisting of SEQ ID No.1, SEQ ID No.2, SEQ ID No.3 e SEQ ID No.4.


SEQUENCE LISTING (6 sequences)

SEQ ID NO: 1 - protein, 9 aa, Artificial Sequence
  exposed protein
QRKLAAKLT

SEQ ID NO: 2 - protein, 9 aa, Artificial Sequence
  exposed protein
RVRSAPSSS

SEQ ID NO: 3 - protein, 9 aa, Artificial Sequence
  exposed protein
RKILRAGPL

SEQ ID NO: 4 - protein, 9 aa, Artificial Sequence
  exposed protein
SWSCTRYAA

SEQ ID NO: 5 - DNA, 17 bp, Artificial Sequence
  reverse primer
gttttcccagtcacgac

SEQ ID NO: 6 - DNA, 23 bp, Artificial Sequence
  forward primer
gctaccctcgttccgatgctgtc